(19) United States
(12) Patent Application Publication
(10) Pub. No.: US 2003/0083605 A1
(43) Pub. Date: May 1, 2003

(54) Comfortable joint sleeve

(76) Inventor: Allan G. Edmund, Northfield, NJ (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/001,301

(22) Filed: Nov. 1, 2001

(51) Int. Cl. A61F 13/00

(52) U.S. Cl. 602/60

(57) ABSTRACT

An orthopedic sleeve for supporting a joint of a limb of a human patient has an external face, an internal face and two lateral faces. The sleeve is in the form of a tube with an open upper end and an open lower end. The sleeve has a first section with a layer of a stiff elastomer and a second section with a panel of stretchable, breathable fabric. The second section is connected to the first section along a pair of seams, which extend lengthwise along the limb of the patient adjacent to the lateral faces of the joint. The first and second sections are connected via the use of an overlock or flat stitch, which allows for a smooth interior surface of the sleeve. The sleeve can also shaped to conform to the natural bend of a joint via cutting sections of the first section of the sleeve, and connecting the severed edges via a zig-zag stitch to form two seams. By doing so, a sleeve is provided which imparts warmth and compression to the external face of the joint, while providing coolness and breathability to the internal face of the joint.

### SUMMARY OF THE INVENTION

This invention relates to a sleeve for a joint of a limb of a human patient, especially a knee or elbow, and particularly to a sleeve that imparts warmth and compression to the external face of the joint where needed, and breathability and coolness to the internal face of the joint, providing optimal comfort, as well as security while the limb is straight or bent.

Participation in daily exercise is recommended by physicians in practically every medical field, and an increasing segment of the population is partaking in physical activities. While consultation with a physician before beginning an exercise regime is suggested, that suggestion is not always followed. Therefore, injuries to joints, especially the knee and elbow, as a result of exercise has become more common. Many injuries to the knee render a person immobile or restrict the person's activities for long periods of time.

Once a joint has been injured, further injury becomes commonplace, necessitating either surgical correction or physical therapy sessions to improve the strength of the joint, and to alleviate pain. These procedures are time consuming and expensive, and are not always successful in correcting the injury or removing the pain.

The prevalence of injuries associated with joints due to sports-related activities, everyday type activities, and congenital disorders has provided the foundation for the use of sleeves. Sleeves have been used to provide support to injured joints, by restricting the joint from extended movement, or have alternatively been used to prevent injury from occurring in the first place. Further, sleeves are worn by those with injured joints in order to circumvent either minor surgery or lifelong physical therapy sessions.

A variety of sleeves are available for such a purpose, either over-the-counter or through prescription from a physician, and include those sleeves described in U.S. Pat. Nos. 3,092,110, 3,613,681, 3,677,265, 4,084,584 and 4,474,573. However, while the sleeves currently available may provide adequate support, they tend to be uncomfortable.

Many of the sleeves described in the prior art have been constructed from multiple pieces of a material having a single layer and having vertical and horizontal seams, which intersect in the back. The intersecting seams can bunch-up, causing discomfort, pain, and sometimes sores, when the joint is left in the flexed position, such as in sitting, or when the joint is rapidly and repeatedly flexed when partaking in physical activities.

Not only does the sleeve have a tendency to impart pain and discomfort, and to cause sores at the internal face of the joint as a as a result of the ill-placed seam, but the material used for support in these sleeves tends to be thick and air-and water-impenetrable. In the sleeves utilized up until now, this thick and impenetrable material is used to form the entire sleeve, including the internal face of the joint. Because the internal face of the joint tends to be softer and is easily irritated, it becomes hot, moisture tends to accumulate, and frequently starts to itch. Many times this necessitates removal of the sleeve and cleaning off the internal face of the joint, only to replace the sleeve and repeat the process.

The sleeves described in the prior art attempt to remedy the aforementioned problems associated with discomfort, particularly addressing problems associated with the knee, due to bunching of the sleeve. However, the problems encountered when utilizing an un-breathable fabric covering the internal face of the joint are not addressed.

The joint sleeve of the present invention eliminates all of the above-mentioned problems encountered with the sleeves found in the art.

As used herein with reference to a joint or to the orthopedic sleeve, the term “external” refers to the region anterior to the knee or posterior to the elbow; the term “internal” refers to the region posterior to the knee or anterior to the elbow; and “lateral” refers to regions to either side of the knee or elbow.

In accordance with the invention, an orthopedic sleeve for supporting a joint of a limb of a human patient is provided, where the sleeve has an external face, an internal face, and two lateral faces, and is in the form of a tube having an open upper end and an open lower end. The first section includes a layer of a stiff elastomer, while the second section includes a panel of stretchable, breathable fabric. The second section is connected to the first section along a pair of seams adapted to extend lengthwise along the limb of the patient adjacent to the lateral faces of the joint.

In a preferred embodiment of the invention the open upper end of the sleeve is larger than the open lower end of the sleeve.

The stiff elastomer of the orthopedic sleeve can be a foam neoprene elastomer or blend that is solid, or perforated so that it allows some breathability. The stiff elastomer can be bonded to a stretchable fabric on the internal face of the sleeve, or alternatively sandwiched between stretchable fabric layers. The fabric layer or layers can be bonded to the stiff elastomer by an adhesive or flame laminated (as with a thermoplastic foam).

The stretchable fabric of the orthopedic sleeve can be a synthetic fabric, such as a nylon or polyester fabric. The stretchable, breathable fabric can be elastic, segmented polyurethane fibers, or a material made from yarns including an elastic segmented polyurethane fiber and at least one material selected from the group consisting of nylon, polyolefin, cotton, and polyester.

In a preferred embodiment of the invention, the seams of the orthopedic sleeve are stitched thread, forming a flat lock or overlook stitch on the outside of the sleeve, to provide a smooth interior surface in the sleeve.

In a preferred embodiment of the invention, the first section has a first seam extending from a point adjacent to the upper end lengthwise along a midline of the first section to a point above a midpoint between the upper and lower end, and a second seam extending from a point adjacent to the lower end lengthwise along said midline to a point below the midpoint. Each of the first and second seams is formed by connecting together the edges of a V-shaped notch formed in the first section when flat. By doing so, the first section forms a convex exterior having a compound curvature conforming to the natural bend of the joint. The first and second seams formed by connecting the edges of the V-shaped notch or notches are preferably positioned on the neutral axis of the joint where minimum movement of the joint occurs.

Other objects, details, advantages and modifications of the invention will be apparent from the following detailed description when read in conjunction with the drawings.

### BRIEF DESCRIPTION OF THE DRAWINGS

FIG. 1 is a perspective view of the joint sleeve of this invention, looking from the side thereof.

FIG. 2 is a front elevational view of the joint sleeve of this invention showing the joint schematically.

FIG. 3 is a side elevational view of the joint sleeve of the invention as shown in FIG. 2.

FIG. 4 is an enlarged sectional view taken on the plane 4-4 of FIG. 2.

FIG. 5 is a front elevational view of the joint sleeve of the invention, prior to formation of the seams of FIG. 2.

FIG. 6 is a front elevational view of the joint sleeve, illustrating another embodiment of the joint sleeve of the invention.

FIGS. 7A and 7B are schematic views of the “V” shaped notches as defined in the joint sleeve of the invention.

FIG. 8 is a front elevational view of the joint sleeve of the invention, illustrating a further embodiment.

### DETAILED DESCRIPTION

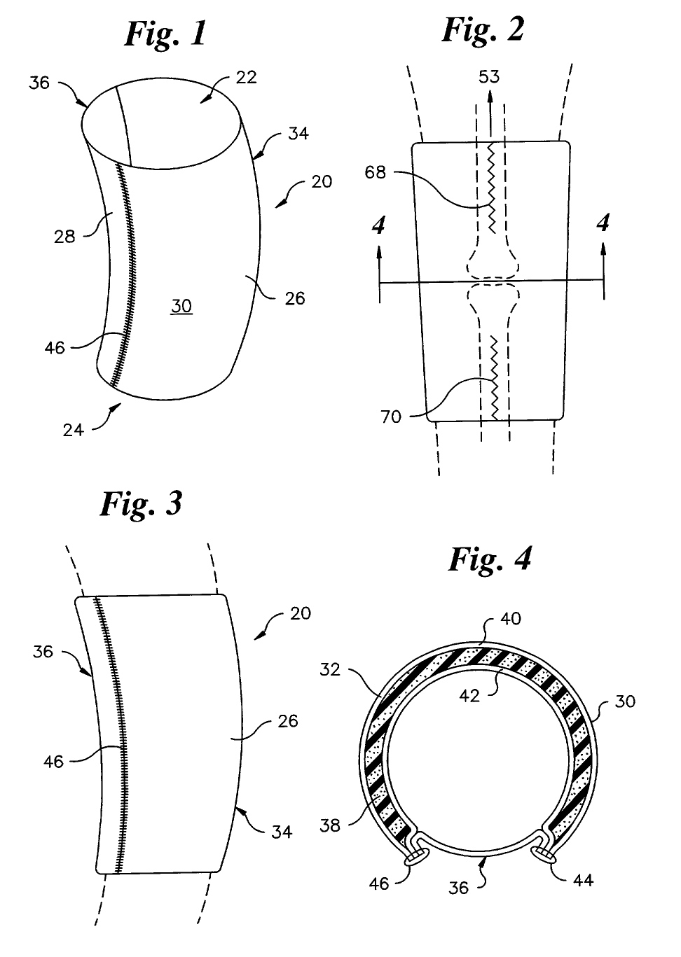

A sleeve 20 in accordance with the invention is shown in FIG. 1. The sleeve is in the form of a tube having an open upper end 22 and an open lower end 24. The sleeve 20 has an external face 26, an internal face 28, and two lateral faces 30 and 32 (FIG. 4). The sleeve 20 is composed of a first section 34 and a second section 36, which are connected together to form the tube.

Sections 34 and 36 are made from different materials. In the embodiment illustrated in the drawings section 34 comprises a layer of stiff elastomer 38 (FIG. 4) and coverings, composed of stretchable fabric layers 40 and 42, on its exterior and interior faces, respectively.

The presence of the stretchable fabric on the interior of the first section 34 of the sleeve 20 depends on the needs of the patient. The layer of stretchable fabric on the interior of the first section 34 of the sleeve 20, enables the sleeve 20 to be slid into place easily and comfortably. Alternatively, if more security or support is required, a sleeve without fabric on the interior of the first section 34 may be utilized. A sleeve without an interior fabric layer is more difficult to slide into place, but is more secure and provides more resistance to movement during flexion. For sleeves in which no stretchable fabric is placed on the interior, a variety of known lubricating powders can be utilized to aid the application of the sleeve. Regardless of whether or not the stretchable fabric is placed on the interior, the exterior covering of stretchable fabric protects the stiff elastomer 38 from deterioration as a result of abrasion.

The stiff elastomer 38 should be durable, stretchable, and provide warmth and support to a joint. A variety of stiff elastomers are known in the art including foamed elastomers such as rubber, neoprene, latex, and polyurethane, among others. The required thickness of the stiff elastomer 38 depends upon the amount of resistance necessary. However, standard thicknesses of about 1/16 of an inch to about ¼ of an inch are generally employed.

The stretchable fabric layers 40 and 42, which cover the stiff elastomer 38 can be made from synthetic fabrics, such as fabrics made from nylon or polyester yarns. The fabrics are preferably made by winding combinations of different fibers into a yarns and using the yarns to form the stretchable fabric layers 40 and 42. The synthetic fibers, impart durability and abrasion resistance to the sleeve 20. However, natural fibers such as cotton can be incorporated into the synthetic fabric yarns of the stretchable fabric. The stretchable fabric is generally of standard thickness wherein a piece of fabric about 60 inches wide weighs about 3 ounces to about 16 ounces per lineal yard. However, the stretchable fabric can be thinner or thicker depending upon the materials utilized and the desired degree of protection provided to the stiff elastomer 38.

The stretchable fabric can be attached to the stiff elastomer 38 by a variety of techniques including the use of adhesives and flame lamination. A variety of adhesives may be utilized, including both solvent and water-based adhesives. Similarly, a variety of techniques in addition to adhesion and flame lamination may be used to bind the stretchable fabric layers 40 and 42 to the stiff elastomer 38.

The second section 36 of the sleeve is a panel composed of stretchable, breathable fabric (FIG. 4). The term “breathable” as used herein with reference to a fabric means that the fabric is sufficiently thin and permeable to provide coolness to the joint and allows excess moisture to dissipate without accumulating at the internal face of the joint. The fabric of the second section 36 must be thick enough to provide support on the internal face of the joint, but must not be so thick that it bunches behind the joint when flexed. Typically, the stretchable, breathable fabric comprises elastic fibers or a yarn including the same. A variety of polyurethane fibers known to those skilled in the art may be used. Suitable, commonly available fibers, are the segmented polyurethane fibers available from E.I. DuPont de Nemours and Company of Wilmington, Del. under the trademark LYCRA. Other suitable yarns known to those skilled in the art are made from natural and synthetic fibers including nylon, polyolefins, cotton, and polyester, among others. The second section 36 can alternatively be in the form of a net or a weave, made from interlocking fibers or yarns, which, by virtue of its open structure, allows for the rapid evaporation of excess moisture. The breathable fabric is generally of standard thickness wherein a section of fabric about 60 inches wide weighs about 3 ounces to about 16 ounces per lineal yard. However, the breathable fabric may be thinner or thicker. If more support on the internal face of the joint is needed thicker sections, or sections made from several layers of the breathable fabric, may be utilized, while still maintaining the coolness and breathability characteristics of the breathable fabric section.

The joint sleeve 20 is assembled by connecting the first section 34 to the second section 36 along the full lengths of their vertical edges to form seams 44 and 46. The seams thereby extend along each side of the sleeve 20, as illustrated in FIG. 4. The seams connecting sections 34 and 36 are constituted by stitching along the adjoining vertical edges of sections 34 and 36. The stitching is carried out using thread, preferably heavy, durable threads that have elastic properties, such as nylon. The stitching can be performed using any type of stitch known to those skilled in the art, but is preferably an overlock stitch or flat stitch, preferably a feed-of-the-arm flat stitch. By using an overlock stitch, the first and second sections 34 and 36 meet to form a smooth surface on the interior of the sleeve 20 in order to avoid, or at least minimize, irritation of the patient's skin. By using a flat stitch, a smooth surface may be formed on one or both of the interior face and exterior face of the sleeve 20. Only one line of stitching is necessary. However further lines of stitching may be employed to improve the strength of the seams 44 and 46, and to prevent unraveling.

In an alternative embodiment, tape (not shown) may be placed over and/or under the seams of sleeve 20. Specifically, tape may be placed over and/or under seams 44 and 46 prior to stitching sections 34 and 36 together to form the sleeve of the invention. By utilizing tape, a smoother and flatter seam is provided, for greater comfort to the patient. A variety of tapes, including sewn-on tapes, may be used.

A particularly noteworthy feature of the invention is the various shapes and curvatures to which the sleeve 20 can be adapted. Unaltered, the sleeve 20 is in the shape of a tube, with both its upper and lower ends 22 and 24 being equal in circumference. This type of sleeve is most useful to patients having slender limbs. Alternatively, for the average patient, whose limb is larger above the joint than below the joint, the upper end 22 should be somewhat larger than the lower end 24.

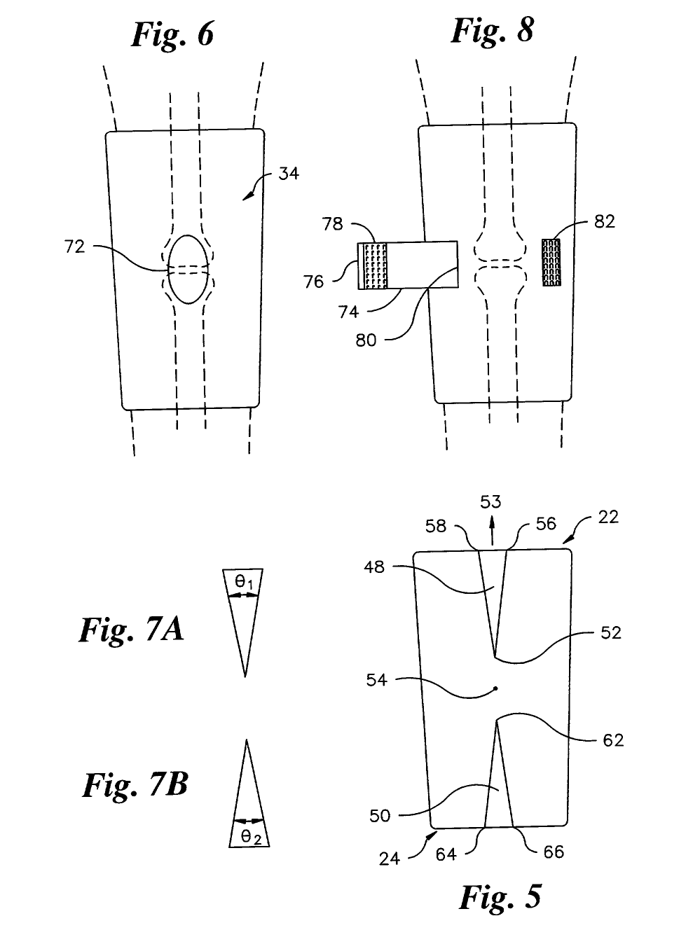

The sleeve 20 can also be preformed to the shape of a curved tube to conform to the natural shape of the joint when the joint is flexed. In this way, further comfort can be achieved during activities where the joint is bent, i.e., during physical activity, or when sitting for prolonged periods of time. The curvature of sleeve 20 is achieved by forming two “V” shaped triangular notches 48 and 50 about line 53 of FIG. 5. The two “V”-shaped notches 48 and 50 are formed by removal of “V”-shaped sections of material adjacent to the upper and lower ends 22 and 24 respectively of the sleeve 20. Notch 48, adjacent to the upper end 22 of the sleeve 20 extends from a point 52, above a midpoint 54 between the upper and lower ends of the sleeve, to points 56 and 58 along the upper edge of the sleeve. Notch 50 similarly extends from point 62, below midpoint 54, to points 64 and 66 on the lower edge of the sleeve.

The sizes of the notches 48 and 50 formed in the first section 34 of the sleeve can vary, and depend on the amount of curvature desired in the final product when in its relaxed condition. The angles θ₁ and θ₂ of the apices at points 52 and 62 the triangular shaped notches 48 and 50, respectively, correspond to the size of the notches 48 and 50 removed (FIG. 5) and will typically range from 0° to about 45°. For example, if sleeve 20 requires a considerable amount of curvature, larger wedges of material are removed to produce more pronounced notches 48 and 50. In this case, the angles (θ₁ and θ₂) formed by the triangular “V” shaped notches 46 and/or 48 are in the upper part of the 0° to about 45° range. If only a slight amount of curvature is necessary, smaller notches 46 and/or 48 are provided. In this situation, the angles (θ₁ and θ₂) formed by the triangular “V” shaped notches 46 and/or 48 are in the lower part of the 0° to about 45° range. In one embodiment, the notches 48 and 50 are equal is size, giving a symmetrical curvature to the sleeve 20. Alternatively, notches 48 and 50 vary in size and impart an asymmetric shape to the sleeve 20. Thus, the brace may be fitted to conform to the exact shape of the joint. These “V” shaped notches 48 and 50 are formed through the use of cutting instruments, such as scissors, razors, or other mechanical cutting devices.

Following the formation of the “V” shaped notches 48 and 50, the vertical edges of the notches are brought together and stitched, to form seams 68 and 70 as shown in FIG. 2. The stitching is carried out with thread, preferably heavy, durable, elastic threads, such as nylon. The stitching can be any type of stitch known to those of skill in the art, but is preferably a zig-zag stitch. The zig zag stitch brings evenly together the severed edges of the notches that form the triangular shape, keeping the first section 34 from puckering and forming unnatural bends. This type of stitch also allows for the formation of a smooth interior face in the sleeve 20. Only one line of stitching is necessary. However, plural lines of stitching can be used to reinforce seams 68 and 70 and to resist unraveling.

One advantage to the sleeve in accordance with the invention is that it can be designed to fit a range of people with small to large limbs. This allows for individuals having limbs of uniform size directly above and below the joint, as well as those with large differences.

Because seams 68 and 70 do not extend along the full length of the first section 34, a convenient area is provided for an opening 72, as shown in FIG. 6. Specifically, the opening 72 allows the joint, a patella if the limb is a knee, or an elbow in the case of an arm, to protrude from the sleeve 20. The provision of the opening reduces friction between the joint and the interior face of the sleeve, and allows the joint to move freely, thereby reducing fluid build-up in the joint. The opening also provides more comfort to the patient during flexion of the joint. However, this embodiment should not be utilized where support is necessary for joints that buckle, or tend to become displaced, i.e., “fall out of place”.

Another embodiment of the joint sleeve of the invention is a sleeve, illustrated in FIG. 8, intended for patients whose knee or knees buckle or are easily displaced. A rectangular-shaped flap 74 is utilized to prevent displacement of a patella. Flap 74 may be prepared from one or more layers of stretchable fabrics, or alternatively may be prepared by sandwiching stiff elastomer 38 between layers of stretchable fabric. The stretchable fabrics corresponding to layers 40 and 42 and the stiff elastomer layer 38 described above, may be utilized to form the flap 74 as well as the first or external layer of the sleeve. Attached to flap 74, and in close proximity to edge 76 of the flap, is a section 78 of a hook and loop fastener. The size of the section 78 can vary and depends on the size of flap 74. Flap 74 is attached to sleeve at its opposite end 80 to one side of the midpoint of the sleeve by an adhesive or by stitching. A mating section 82 of hook and loop fastener material, is attached to the sleeve on the side of the midpoint opposite flap 74. Flap 74 can then be brought across the knee joint and secured to the sleeve to provide additional support to the patella and thereby prevent displacement of the knee. The use of sleeves for restricting patellas from being displaced is further described in U.S. Pat. No. 4,084,584, which is incorporated herein by reference.

It will be apparent from the foregoing description that the advantages afforded by the combination of an external section incorporating a stiff elastomer and an internal section of breathable fabric can be realized in any of a wide variety of sleeve configurations and modifications. Still other modifications in addition to those specifically described may be made to the sleeve described above without departing from the scope of the invention as defined in the following claims.

1. An orthopedic sleeve for supporting a joint of a limb of a human patient, the sleeve having an external face, an internal face and two lateral faces, the sleeve being in the form of a tube having an open upper end and an open lower end and further comprising:
a first section comprising a layer of a stiff elastomer; and
a second section comprising a panel of stretchable, breathable fabric;
wherein the second section is connected to the first section along a pair of seams adapted to extend lengthwise along the limb of the patient adjacent to the lateral faces of the joint.

2. The orthopedic sleeve according to claim 1, wherein the open upper end of the sleeve is larger than the open lower end of the sleeve.

3. The orthopedic sleeve according to claim 1, wherein the stiff elastomer is a foam neoprene elastomer.

4. The orthopedic sleeve according to claim 1, wherein the stiff elastomer is bound to a stretchable fabric on the internal face of the sleeve.

5. The orthopedic sleeve according to claim 4, wherein the stretchable fabric is a synthetic fabric.

6. The orthopedic sleeve according to claim 5, wherein the synthetic fabric layer comprises a nylon or polyester fabric.

7. The orthopedic sleeve according to claim 1, wherein the stiff elastomer is sandwiched between stretchable fabric layers.

8. The orthopedic sleeve according to claim 7, wherein the stretchable fabric layers are bonded to the stiff elastomer by an adhesive.

9. The orthopedic sleeve according to claim 1, wherein the stretchable, breathable fabric comprises elastic, segmented polyurethane fibers.

10. The orthopedic sleeve according to claim 1, wherein the stretchable, breathable fabric layers comprises segmented polyurethane fibers.

11. The orthopedic sleeve according to claim 1, wherein the stretchable, breathable fabric is a material comprising yarns including an elastic segmented polyurethane fiber and at least one material selected from the group consisting of nylon, polyolefin, cotton, and polyester.

12. The orthopedic sleeve according to claim 1, wherein the seams are stitched thread, forming a stitch on the outside of the sleeve, whereby the first and second sections form a smooth interior surface in the sleeve.

13. The orthopedic sleeve according to claim 1, wherein the internal face of the sleeve is substantially smooth and uninterrupted from a location adjacent the upper end of the tube to a location adjacent the lower end of the tube.

14. The orthopedic sleeve according to claim 1, wherein the first section has a first seam extending from a point adjacent to said upper end lengthwise along a midline of the first section to a point above a midpoint between the upper and lower end, and a second seam extending from a point adjacent to said lower end lengthwise along said midline to a point below said midpoint.

15. The orthopedic sleeve according to claim 14, wherein each of the first and second seams is formed by connecting together the edges of a V-shaped notch formed in the first section when flat.

16. The orthopedic sleeve according to claim 15, wherein the first section has a convex exterior having a compound curvature conforming to the natural bend of the joint.

17. An orthopedic sleeve for supporting a joint of a limb of a human patient, the sleeve having an external face, an internal face and two lateral faces, the sleeve being in the form of a tube having an open upper end and an open lower end and further comprising:
a first section comprising a layer of a stiff elastomer sandwiched between stretchable fabric layers; and
a second section comprising a panel of stretchable, breathable fabric;
wherein the second section is connected to the first section along a pair of seams adapted to extend lengthwise along the limb of the patient adjacent to the lateral faces of the joint, and
wherein the internal face of the sleeve is substantially smooth and uninterrupted from a location adjacent the upper end of the tube to a location adjacent the lower end of the tube.

18. An orthopedic sleeve for supporting a joint of a limb of a human patient, the sleeve having an external face, an internal face and two lateral faces, the sleeve being in the form of a tube having an open upper end and an open lower end and further comprising:
a first section comprising a layer of a stiff elastomer sandwiched between stretchable fabric layers and having a first seam extending from a point adjacent to said upper end lengthwise along a midline of the first section to a point above a midpoint between the upper and lower end, and a second seam extending from a point adjacent to said lower end lengthwise along said midline to a point below said midpoint, each of the first and second seams connecting together the edges of a V-shaped notch formed in the first section when flat; and
a second section comprising a panel of stretchable, breathable fabric;
wherein the second section is connected to the first section along a pair of seams adapted to extend lengthwise along the limb of the patient adjacent to the lateral faces of the joint, and wherein the internal face of the sleeve is substantially smooth and uninterrupted from a location adjacent the upper end of the tube to a location adjacent the lower end of the tube.
